SEQUENCE LISTING (5 sequences)

SEQ ID NO: 1 - protein, 15 aa, Tolypocladium niveum
  MISC_FEATURE (1)..(1): ACETYLATION, pipecolic acid
XAXAALAGAAXAGLA

SEQ ID NO: 2 - protein, 15 aa, Tolypocladium niveum
  MISC_FEATURE (1)..(1): ACETYLATION, pipecolic acid
XAXAALAGAAXAGLV

SEQ ID NO: 3 - protein, 15 aa, Tolypolcadium niveum
  MISC_FEATURE (1)..(1): Acetylation, pipecolic acid
XAXVALAGAAXAGLV

SEQ ID NO: 4 - protein, 15 aa, Tolypocladium niveum
  MISC_FEATURE (1)..(1): ACETYLATION, pipecolic acid
XAXAALAGAAXAALV

SEQ ID NO: 5 - protein, 15 aa, Tolupocladium niveum
  MISC_FEATURE (1)..(1): ACETYLATION, pipecolic acid
XAXVALAGAAXAALV

(19) United States
(12) Patent Application Publication
(10) Pub. No.: US 2004/0087489 A1
(43) Pub. Date: May 6, 2004

(54) Compositions and methods for the treatment of mycobacterial infections

(76) Inventors: Antonio Ruiz, Gaithersburg, MD (US); Adonia Papathanassiu, Silver Spring, MD (US)

Correspondence Address: Adonia Papathanassiu, P.O. Box 1001, Silver Spring, MD, 20910, US

(21) Appl. No.: 10/660,051

(22) Filed: Sep. 11, 2003

Related U.S. Application Data
(60) Provisional application No. 60/424,265, filed on Nov. 6, 2002.

(51) Int. Cl. A61K 38/00; A61K 31/7048; A61K 31/715; A61K 31/32

(52) U.S. Cl. 514/2; 514/27; 514/54; 514/493; 514/422; 514/456

(57) ABSTRACT

The invention relates to composition and methods for the treatment of Gram-positive bacterial infections. More specifically, the invention describes the use of ATP synthase and vacuolar ATPase inhibitors for the treatment of mycobacterial infections particularly tuberculosis.

### CROSS-REFERENCE TO RELATED APPLICATIONS

The present nonprovisional patent application claims benefit of provisional patent application entitled “Compositions and Methods for the Treatment of Mycobacterial Infections” with filing date Nov. 6, 2002 and patent application Ser. No. 60/424,265.

### STATEMENT REGARDING FEDERALLY SPONSORED RESEARCH OR DEVELOPMENT

Not applicable

### REFERENCE TO A SEQUENCE LISTING

Not applicable

### BACKGROUND OF THE INVENTION

The mycobacteria are a diverse collection of acid-fast, non-motile, gram-positive bacteria. It comprises several species, which include, Mycobacterium africanum (M. africanum), M. avium, M. bovis, M. bovis-BCG, M. chelonae, M. fortuitum, M. gordonae, M. intracellulare, M. kansasii, M. microti, M. scrofulaceum, M. paratuberculosis, M. leprae, M. tuberculosis, and M. ranae. Certain of these organisms are the causative agents of disease. For example, M. leprae is the causative agent of leprosis, while M. tuberculosis is the causative agent of tuberculosis or TB. In man, M. tuberculosis grows in the endobronchial space and occasionally in the alveoli of infected individuals, where it results in the inflammation and progressive destruction of the lungs, the hallmarks of TB. Other manifestations of the disease include fever and nonproductive cough.

TB is a chronic infectious and highly contagious disease, which can remain aymptomatic and, thus, untreated for considerable periods of time. Untreated active TB may result in serious complications and even death. There are approximately 8 million new cases of active TB every year worldwide and about 2 million fatalities. With the total estimated number of infected individuals reaching 1.86 billion, TB is considered a serious a public problem. It is a major disease in developing countries and in some developed areas of the world, especially sub-Saharan African countries and the newly independent states of the former Soviet Union. Cases of mycobacterial infections have also been reported and considered to be on the rise in the United States and Europe. A large number of the new cases are related to the AIDS epidemic. AIDS-related TB is considered a fatal disease. Immune compromised AIDS patients are also susceptible to non-TB mycobacteria infections like Mycobacterium avium and Mycobacterium kansasii. (Kiehn et al., J. Clin. Microbiol., 21:168-173 (1985); Wong et al., Amer. J. Med., 78:35-40 (1985)).

Tuberculosis is usually controlled using extended antibiotic therapy. There are four front-line drugs, isoniazid (INH), rifampicin (RMP), pyrazinamide (PZA), and ethambutol (EMB), which are highly effective against M. tuberculosis and several second-line drugs including streptomycin (STR), which are used when resistance to one or more of the front-line drugs is detected. During standard treatment, TB-infected individuals receive 2-months of an INH-RPM-PZA combination followed by 4-months of INH-RMP.

Although TB chemotherapy can be highly effective, the duration of the treatment and the side-effects associated with some of the drugs in the regimen adversely affect compliance. Lack of adherence to treatment has been associated with relapse and the rise of drug-resistance. Recent surveys reveal that TB cases caused by organisms resistant to INH and RMP are on the rise in US and worldwide. Outbreaks of multidrug-resistant tuberculosis (MDR-TB) have occurred in various US hospitals and in prisons of independent states of the former Soviet Union. INH-monoresistant tuberculosis is often treated successfully by adding EMB to the INH-RPM-PZA combination, while MDR-TB patients are treated with a combination of second-line drugs, which are significant more toxic and less effective than the first-line drugs.

Although many scientific studies have been directed at diagnosis, treatment and control of this disease, the diagnostic, immunoprophylactic, and treatment methods have changed little in the last fifty years. The only existing vaccine, the Bacillus Calmette-Guerin (BCG) vaccine, has had a limited impact on TB despite its wide use [Calmette, A., Masson et Cie, Paris (1936)]. Some studies have shown that it has protective efficacy against tuberculosis [Luelmo, F., Am. Rev. Respir. Dis., 125, 70-72 (1982)], while, in other studies, BCG has failed to protect against tuberculosis [WHO, Tech. Rep. Ser., 651:1-15 (1980)] for reasons that are not entirely clear [Fine, P., Tubercle, 65:137-153 (1984); Fine, et al., Lancet (ii):499-502 (1986)]. It is generally accepted that BCG vaccine protects the development of some forms of TB in young children, but it is less protective in adults. Recently, new emphasis has been given in the development of a new and effective TB vaccine. Unfortunately, this vaccine is considered a long-term project and it might take up to 25 years to be developed.

It is apparent that what is needed is the development of new, safe and effective antibiotic drugs appropriate to treat classical and MDR-TB with a shortened treatment course and fewer side effects. Traditional TB drugs are mycobacteria-specific and act by inhibiting bacterial metabolism, especially the construction of the cell wall superpolymer. For example, INH interferes with the enzymatic machinery that synthesizes mycolic acids, necessary components of the cell wall, while RMP interferes with the bacterial machinery for transcribing RNA from DNA. Subsequently, it is of great interest to develop drugs with alternative modes of action capable of overcoming drug resistance

### BRIEF SUMMARY OF THE INVENTION

This invention encompasses methods for treatment of infections with Gram positive bacteria, particularly mycobacterial infections, and most particularly those caused by M. africanum, M. avium, M. bovis, M. bovis-BCG, M. chelonae, M. fortuitum, M. gordonae, M. intracellulare, M. kansasii, M. microti, M. scrofulaceum, M. paratuberculosis, M. leprae and M. tuberculosis, and M. ranae.

The methods provided herein for treating mycobacterial infections involve administering to a human or animal a composition containing therapeutic dosages of one or more inhibitors of F₁F₀-ATP synthase or V-ATPase. The nature of the molecule or molecules could be, but not limited to, purified from culture filtrates, synthetically produced or any recombinant produced molecule or fragment. More specifically, the present invention describes methods for treatment of mycobacterial infections utilizing F₁F₀-ATP synthase or V-ATPase inhibitors selected from a group including the natural inhibitor of F₁F₀-ATP synthase (IF₁), aurovertins, citreoviridin, citreoviridin acetate, quercetin, oligomycins, peliomycin, N,N′-Dicyclohexylcarbodiimide, venturicidins, trimethyl tin chloride, triethyl tin chloride, tri-n-propyl tin chloride, tri-n-butyl tin chloride, triphenyl tin chloride, DBCT, ossamycin, leucinostatin, and especially efrapeptins.

### BRIEF DESCRIPTION OF DRAWINGS

FIG. 1A is a schematic diagram showing the chemical structure of oligomycin A. FIG. 1B is a schematic diagram showing the chemical structure of oligomycin B. FIG. 1C is a schematic diagram showing the chemical structure of oligomycin C.

FIG. 2 is a schematic diagram showing the chemicals structures of aurovertin B, citreoviridin, and α-zearalenol.

FIG. 3 is a schematic diagram showing the sequence and structure of efrapeptins.

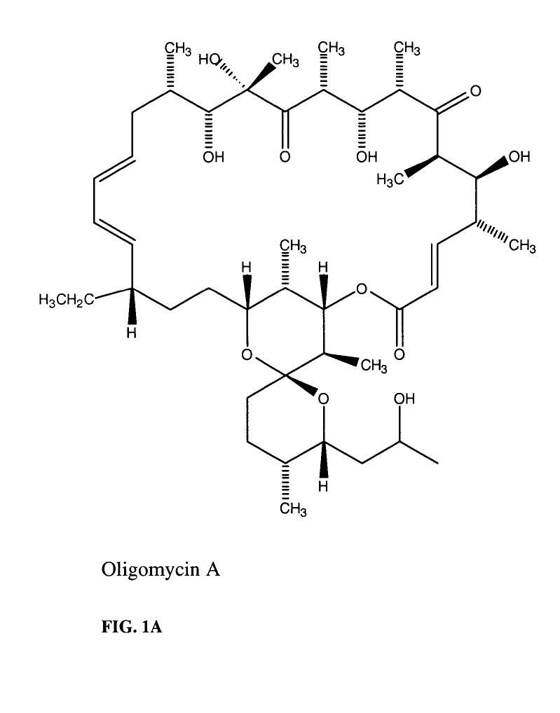

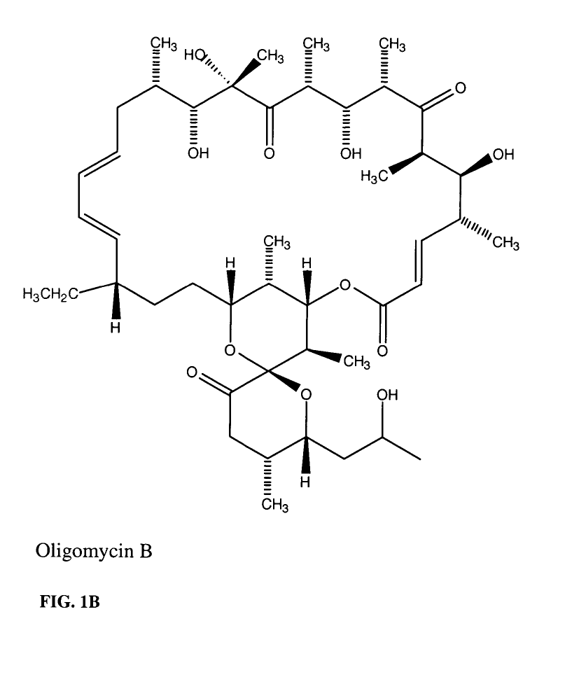

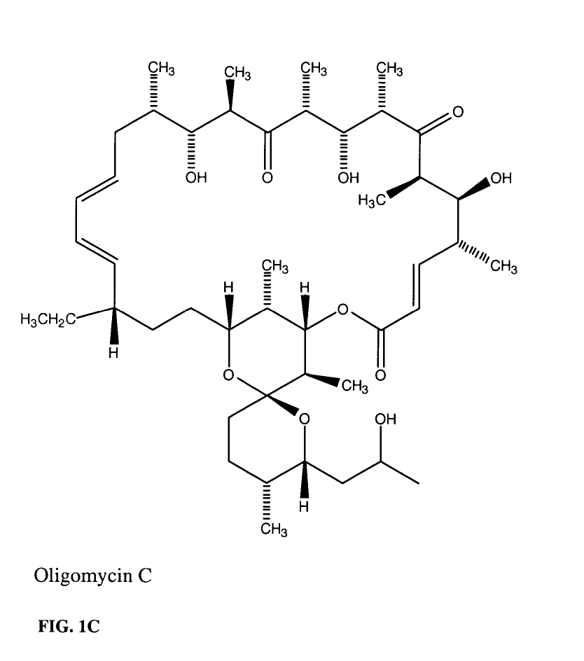

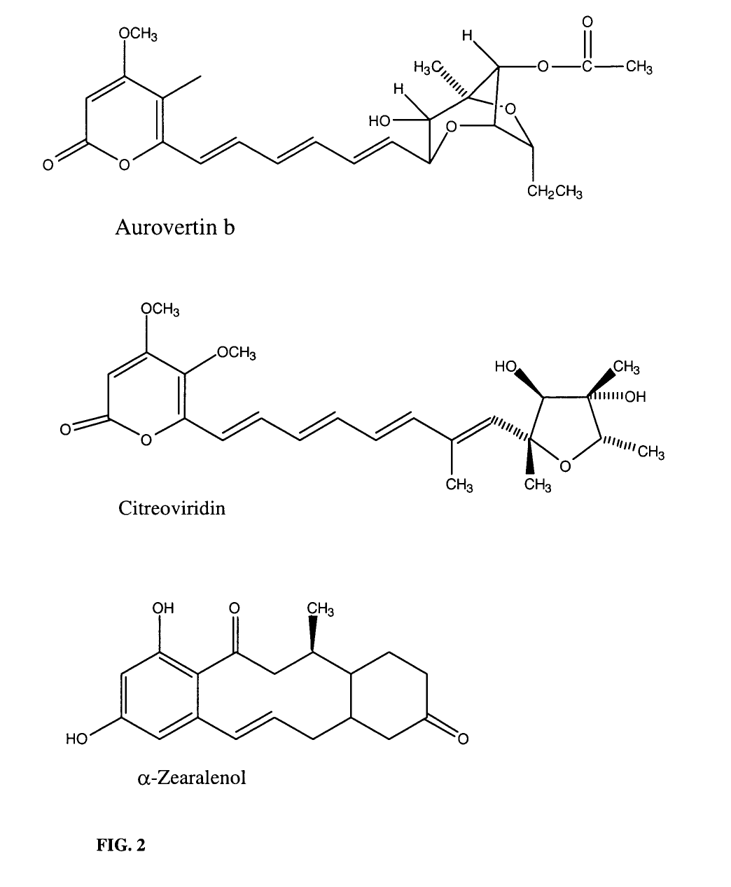

### DETAILED DESCRIPTION OF THE INVENTION

F₁F₀-ATP synthase catalyses the hydrolysis of ATP to ADP and phosphate. The crystal structure of bovine F₁-ATPase has been determined previously to a 2.8 Å resolution. The enzyme comprises five different subunits in the stoichiometry α₃β₃ΓΔε; the three catalytic β-subunits alternate with the three α-subunits around the centrally located single Γ-subunit.

Members of the F₁F₀-family of ATP synthases and V-ATPase are present in bacteria, in chloroplast membranes, and in mitochondria. [Molecular Biology of the Cell, Alberts et al., eds., Garland Publishing, Inc., New York (1983), pages 484-510.] The enzyme is well conserved; the α- and β-subunit polypeptides from different sources show almost 50% sequence identity, while other F₁-subunit polypeptides show more variation. In the conserved regions of the β-subunit, the primary amino acid sequences are identical among tobacco, spinach, maize, bovine, E. coli and S. cerevisiae. [Takeda et al., J Biol. Chem., 260(29):15458-15465 (1985)].

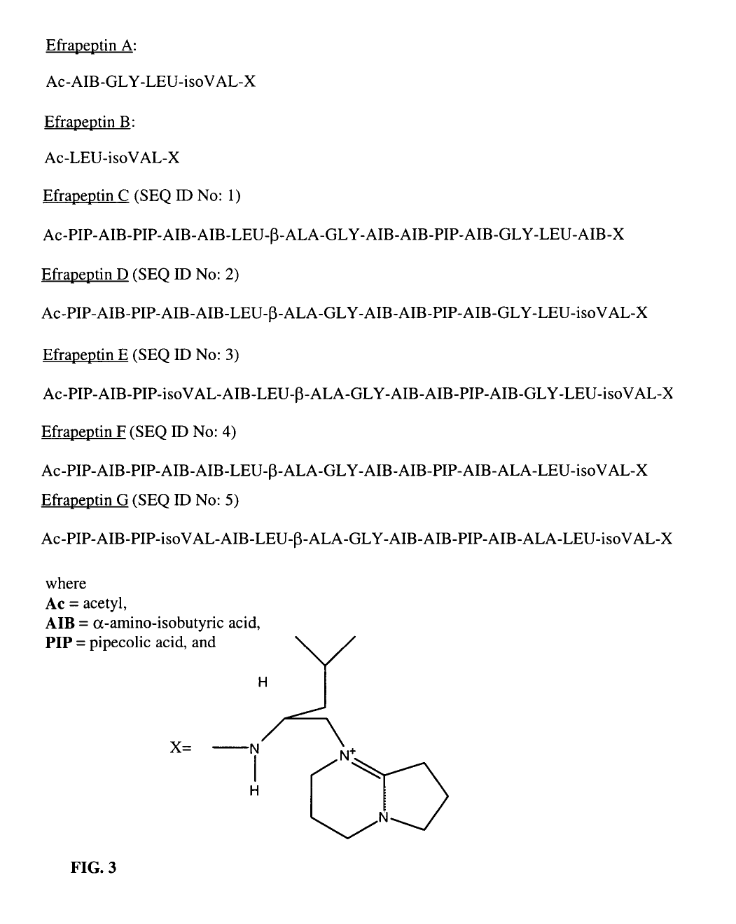

Efrapeptins are a family of apolar, hydrophobic peptides isolated from entomopathogenic fungi and they are known to be potent inhibitors of mitochondrial F₁F₀-ATPase. With the exception of efrapeptin A and B, efrapeptins are composed of 15 amino acids (usually common amino-acids alanine, glycine, leucine and uncommon amino-acids α-aminobutyric acid, β-alanine, isovaline, and pipecolic acid) with the amino-terminal acetylated and the carboxyl-terminal blocked by N-peptido-1-isobutyl-2[1-pyrrole-(1,2-α)-pyrimidinium,2,3,4,5,6,7,8,-hexahydro]-ethylamine [Krasnoff, S. B., et al., Antifungal and Insecticidal Properties of the Efrapeptins: Metabolites of the Fungus Tolypocladium niveum, J. Invert. Path., 58: 180-188 (1991)]. FIG. 3 depicts known efrapeptins.

Efrapeptins inhibit both ATP synthesis and hydrolysis by binding to a unique site in the central cavity of the F₁ catalytic domain of F₁F₀-ATP synthase and inducing a hydrophobic contact with the α-helical structure in the Γ-subunit. It inhibits F₁F₀-ATP synthase activity by blocking the conversion of β-subunit to a nucleotide binding conformation, which is essential for the cyclic interconvertion of the three catalytic sites.

Other inhibitors of F₁F₀-ATP synthase activity include mytotoxins. Mycotoxins are secondary metabolites produced by many pathological and food spoilage fungi including Aspergillus, and Penicillium species. For example, aurovertin B is produced by Calcarisporium Arbuscula, citreoviridin is produced by Penicillium Citreoviride Biourge, while α-zearalenol is produced by Fusarium.

The present invention further provides methods of using the antibiotics in the treatment and prevention of mycobacterial infections and inflammation.

### I. Definitions

The term “reduction or inhibition of mycobacterial infections” is defined as improvement in disease prognosis as indicated by the clinical symptoms in a subject. This benefit is indicative of decrease on inflammation of the lungs, fever and cough. A reduction or inhibition of mycobacterial infections can be indicated by a decrease in the bacterial numbers harvested from lungs and spleens of infected mice.

The terms “F₁F₀-ATP synthase inhibitors” and “V-ATPase inhibitors” are defined as molecule or molecules capable of inhibiting the enzymatic activity of F₁F₀-ATP synthase and V-ATPase, respectively. In a particular embodiment, the antibiotic peptides can act with another antibiotic, such as penicillin, to synergistically reduce or inhibit mycobacterial infections.

The term “antimicrobial drugs” is defined as a molecule capable of inhibiting the growth of or killing mycobacteria. The term “antibiotic peptides” is defined as peptides capable of inhibiting the growth of or killing mycobacteria. Antimicrobial drugs and antibiotic peptides can be administered in a pharmaceutically acceptable carrier. Such administration can be performed topically, by injection, or orally.

The peptides or peptide fragments of the present invention can be purified from culture filtrates, prepared by recombinant means, proteolytic digestions, or preferably chemical synthesis. Analogs or peptide fragments of the peptides can contain portions of the amino acid sequence encoded by the open reading frame alone, or alternatively a portion of the amino acid sequence can be linked together in a fusion peptide. Thus, modification of the peptides of the present invention can also be made in order to make the peptide more stable, more potent or less toxic.

### II. Suitable Methods for Practicing the Invention

Inhibition of M. ranae

The ability of antimicrobial drugs to suppress growth of 1×10⁴ CFU/ml of M. ranae in cultures grown under controlled conditions is evaluated using a standard optical density curve to determine the final inoculum concentration. After four days, growth of the culture is examined and scored positive (+) for inhibition of growth or turbidity or negative (−) for no effect. Minimal inhibitory concentration (MIC) is subsequently determined by standard dilution techniques.

Inhibition of M. tuberculosis

The ability of antimicrobial drugs to suppress growth of 1×10⁴ CFU/ml of M. tuberculosis in cultures grown under controlled conditions is evaluated using the Microplate Alamar Blue Assay (MABA) (Collins et al. Antimicrob. Agents Chemother 41:1004-9 (1997)). Briefly, antimicrobial activity is tested by adding various concentrations of drugs to clear-bottomed, 96-well plates followed by 5×10³ CFU BACTEC 12B-passaged inocula. After an initial incubation at 37° C. for 4 days, Alamar Blue solution is added to the wells and the plates are re-incubated. Fluorescence is measure 12 to 24 hrs later. Minimal inhibitory concentration (MIC) is subsequently determined by standard dilution techniques.

Murine Aerosolized TB Model

Mice are infected with a low-dose aerosol of M. tuberculosis, which deposits approximately 50 bacilli into the lungs of the animals. Treatment is initiated on day 20 post inoculation and is terminated 4 weeks later. Antimicrobial activity is determined at midpoint and at the end of treatment by aseptically dissecting the lungs and spleens and plating whole-organ homogenates on nutrient 7H11 agar and assessing bacterial colony formation at 37° C. in humidified air.

### III. EXAMPLES

Inhibition of M. ranae by efrapeptin D (SEQ ID NO: 2)

The ability of efrapeptin D (SEQ ID NO: 2) to suppress growth of 1×10⁴ CFU/ml of M. ranae (ATCC 110) in cultures grown under controlled conditions was evaluated using a standard optical density curve to determine the final inoculum concentration (MDS Pharma Services, Bothell, Wash.). The experiment was performed in duplicate. After four days, growth of the culture was examined and scored positive (+) for inhibition of growth or turbidity or negative (−) for no effect. Results are shown on Table I. MIC was 18 μM.

TABLE I
Inhibition of M. ranae by Efrapeptin D (SEQ ID NO: 2)

| Concentration in μM | Results |
|---|---|
| 60 | + |
| 18 | + |
| 6 | − |
| 1.8 | − |
| 0.6 | − |
| 0.18 | − |
| 0.6 | − |

Inhibition of M. phlei by efrapeptin D (SEQ ID NO: 2)

The ability of efrapeptin D (SEQ ID NO: 2) to suppress growth of 1×10⁴ CFU/ml of M. phlei (ATCC 11758) in cultures grown under controlled conditions was evaluated using a standard optical density curve to determine the final inoculum concentration (MDS Pharma Services, Bothell, Wash.). The experiment was performed in duplicate. After four days, growth of the culture was examined and scored positive (+) for inhibition of growth or turbidity or negative (−) for no effect. Results are shown on Table II. MIC was 0.6 μM.

TABLE II
Inhibition of M. phlei by Efrapeptin D (SEQ ID NO: 2)

| Concentration in μM | Results |
|---|---|
| 60 | + |
| 18 | + |
| 6 | + |
| 1.8 | + |
| 0.6 | + |
| 0.18 | − |
| 0.6 | − |

We claim:

1. A method of treating a Gram-positive bacterial infection in a human or animal comprising administering to the human or animal a therapeutically active dosage of F₁F₀-ATP synthase inhibitor.

2. The method of claim 1 where the Gram-positive bacterial infection is an infection caused by the group of bacteria including M. africanum, M. avium, M. bovis, M. bovis-BCG, M. chelonae, M. fortuitum, M. gordonae, M. intracellulare, M. kansasii, M. microti, M. scrofulaceum, M. paratuberculosis, M. leprae, M. tuberculosis, and M. ranae.

3. The method of claim 2 wherein the F₁F₀-ATP synthase inhibitor is selected from a group including, but not limited to, IF₁, aurovertins, citreoviridin, citreoviridin acetate, quercetin, oligomycins, peliomycin, N,N′-Dicyclohexylcarbodiimide, venturicidins, trimethyl tin chloride, triethyl tin chloride, tri-n-propyl tin chloride, tri-n-butyl tin chloride, triphenyl tin chloride, DBCT, ossamycin, leucinostatin, and efrapeptins.

4. The method of claim 3 where efrapeptins are selected from a group including, but not limited to oligopeptides with SEQ ID NOs: 1, 2, 3, 4, 5.

5. The method of claim 1 wherein the F₁F₀-ATP synthase inhibitor binds to F₁F₀-ATP synthase.

6. The method of claim 1 wherein the F₁F₀-ATP synthase inhibitor is capable of blocking the enzymatic activity of mitochondrial ATP synthase.

7. The method of claim 1 wherein the F₁F₀-ATP synthase inhibitor is purified from culture filtrates, prepared by any recombinant means, proteolytic digestions, or chemical synthesis.

8. The method of claim 1 wherein analogs or peptide fragments of F₁F₀-ATP synthase inhibitor containing portions of the amino acid sequence are prepared by any recombinant means, proteolytic digestions, or chemical synthesis.

9. The method of claim 1 wherein the F₁F₀-ATP synthase inhibitor is capable of inhibiting the growth of or killing mycobacteria in a human or animal.

10. The method of claim 1 wherein the F₁F₀-ATP synthase inhibitor can be administered with another antibiotic, to synergistically reduce or inhibit mycobacterial infections.

11. A method of treating a Gram-positive bacterial infection in a human or animal comprising administering to the human or animal a therapeutically active dosage of a composition designated as V-ATPase inhibitor.

12. The method of claim 11 where the Gram-positive bacterial infection is an infection caused by the group of bacteria including M. africanum, M. avium, M. bovis, M. bovis-BCG, M. chelonae, M. fortuitum, M. gordonae, M. intracellulare, M. kansasii, M. microti, M. scrofulaceum, M. paratuberculosis, M. leprae, M. tuberculosis, and M. ranae.

13. A method for determining whether a molecule inhibits the growth of Gram positive bacteria in a mammal by inhibiting the enzymatic activity of F₁F₀-ATP synthase, the method comprising of the a screening assay in which the possible inhibition of F₁F₀-ATP synthase by the molecule is determined by adding the substance to a system comprising immobilized F₁F₀-ATP synthase and soluble ATP, enzymatic activity detected by coupling the production of ADP to the oxidation of NADH via pyruvate kinase and lactate hydrogenase reactions.
